(19) United States
(12) Patent Application Publication
(10) Pub. No.: US 2012/0059468 A1
(43) Pub. Date: Mar. 8, 2012

(54) SYSTEMS AND METHODS FOR REPAIR OF A SOFT TISSUE TO BONE ATTACHMENT

(76) Inventors: Ralph MATTERN, San Diego, CA (US); Jamal RUSHDY, Eden Prairie, MN (US); Dale PETERSON, La Jolla, CA (US); Kevin OHASHI, Jamaica Plain, MA (US); Justin C. ANDERSON, Minnetonka, MN (US)

(21) Appl. No.: 13/039,011

(22) Filed: Mar. 2, 2011

Related U.S. Application Data
(60) Provisional application No. 61/309,799, filed on Mar. 2, 2010.

(30) Foreign Application Priority Data
May 7, 2010 (FR) 1053601

(51) Int. Cl. A61F 2/08

(52) U.S. Cl. 623/13.14

(57) ABSTRACT

A strap for repairing the attachment between a bone and a soft tissue according to embodiments of the present invention includes an elongated flexible textile body including, along its longitudinal direction, first and second longitudinal parts, the first longitudinal part including a fixation mechanism, the fixation mechanism configured to mechanically cooperate with the second longitudinal part, after the second longitudinal part has been passed through at least the soft tissue, to form a loop around at least a portion of the soft tissue, wherein the fixation mechanism permits tightening of the loop to tension the elongated flexible textile body to hold the soft tissue in place with respect to the bone.

### CROSS-REFERENCE TO RELATED APPLICATIONS

This application claims the benefit of U.S. Provisional Patent Application Ser. No. 61/309,799, filed on Mar. 2, 2010, and claims foreign priority to French Patent Application Number 1053601, filed on May 7, 2010, both of which are incorporated herein by reference in their entireties for all purposes.

### TECHNICAL FIELD

Embodiments of the present invention relate generally to a repair strip for attaching bone to soft tissue, such as tendon or ligament, and to methods of restoration of such attachment.

### BACKGROUND

When a soft tissue of a human, such as a tendon or a ligament, is damaged at the point at which it attaches to the bone, the attachment may be restored in various ways, such as with sutures and screws. The corresponding surgical procedure is often long and delicate to implement. Despite the care exercised by the surgeon during the procedure, the risks of separation of the suture, so much more at the level of the soft tissue than the bone, are often elevated because, very often, the means used to join the tissue with the bone weakens the soft tissue and/or the bone. This fact is even more critical in the context of restoring the attachment of tendons and ligaments of a rotator cuff to a shoulder bone of a human being, due to the strong mechanical forces attained during shoulder movements.

Arthroscopic repair of rotator cuff tendons is difficult and repairs often fail when the sutures pull through the tendon, or, less often, through the bone tunnel. Use of a patch to reinforce the repairs generally adds considerable time and effort to the surgical procedure. Patch repairs are typically done in open or “mini-open” surgery, and a small number of surgeons who do patch repairs arthroscopically often add forty to ninety minutes to each surgery.

### SUMMARY

Embodiments of the present invention include a band or strip and a method of repair which permit the retention of a soft tissue face-to-face with a bone in a manner that is reliable and durable. Embodiments of the present invention include a band or strip to attach the bone to the soft tissue. Such a band permits performance of a repair method for attaching bone to soft tissue, such as a tendon or a ligament, in which the surgeon places a flexible, elongated textile body, including first and second parts along its longitudinal direction, through soft tissue and bone. The first longitudinal part may include a fixation mechanism, and at least a portion of the second longitudinal part may be passed through the soft tissue and into proximity with the bone, such as for example passed through a tunnel formed in the bone. The second longitudinal part may be placed into mechanical cooperation with the fixation mechanism to form a loop encircling a part of the soft tissue, as well as part of the bone, which may be tensioned to retain the soft tissue face-to-face with respect to the bone.

According to some embodiments of the present invention, the textile strip may be connected to the soft tissue by forming the soft body of the textile strip into a loop threaded through the soft tissue and closed on itself by the fixation mechanism which retains the soft body under tension. In this way, the integration between the body of the strip and the soft tissue is accomplished in a manner which supports the heightened tension forces and effectively transmits them to the soft tissue. In this way, elements such as sutures are not necessary to hold the textile strip in place, according to embodiments of the present invention. Such a loop can even be passed through a bone tunnel, in order to connect the soft tissue and bone in a way that does not require screws, according to embodiments of the present invention.

In practice, the fixation mechanism according to embodiments of the present invention permits the loop formed by the flexible body of the textile strap to be maintained under tension. This loop may take many forms. For example, the flexible body of the textile strap may be formed in various textile materials, in the broadest sense of the term, permitting tissue in-growth and colonization. Due to the quality of the attachment of the strap to the soft tissue and to the bone, the flexible body of the strap can be, depending on the material or materials of which it is constructed, stronger and stiffer than the tissue being repaired.

A method for attaching soft tissue to bone according to embodiments of the present invention includes passing a flexible elongated textile body through a portion of the tissue, the flexible elongated textile body including a fixation mechanism, connecting the flexible elongated textile body onto itself with the fixation mechanism to form a loop, engaging the flexible elongated textile body with the bone, and narrowing the loop to tension the flexible elongated textile body to maintain the portion of the tissue in place with respect to the bone.

In some cases, the flexible elongated textile body has an ultimate stress property higher than the tissue. Engaging the flexible elongated textile body with the bone may include passing the flexible elongated textile body through a bone tunnel in the bone. Passing the flexible elongated textile body through the bone tunnel may include passing the flexible elongated textile body through the bone tunnel before the loop is formed. Passing the flexible elongated textile body through the tissue may include passing the flexible elongated textile body through the tissue a first time, and the method may further include passing the flexible elongated textile body through the tissue a second time. According to some embodiments of the present invention, passing the flexible elongated textile body through the tissue includes passing the flexible elongated textile body through the tissue at a first location, wherein the bone tunnel is a first bone tunnel, the method further including passing the flexible elongated textile body through the tissue at a second location and passing the flexible elongated textile body through a second bone tunnel in the bone. According to some embodiments of the present invention, passing the flexible elongated textile body through the tissue at the first and second locations and through the first and second bone tunnels occurs before the flexible elongated textile body is connected onto itself with the fixation mechanism. In some cases, the flexible elongated textile body is a first flexible elongated textile body and the bone tunnel is a first bone tunnel, the method further comprising passing the second flexible elongated textile body through the tissue and through a second bone tunnel in the bone.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a front elevation view of a repair strap according to embodiments of the present invention.

FIG. 2 illustrates a top plan view of a portion of the repair strap of FIG. 1, taken along arrow II of FIG. 1, according to embodiments of the present invention.

FIG. 3 illustrates a front partial cross-sectional view of the fixation mechanism of FIG. 2, taken along lines III-III of FIG. 2, according to embodiments of the present invention.

FIG. 4 illustrates a side view of the fixation mechanism of FIG. 3, taken along arrow IV of FIG. 3, according to embodiments of the present invention.

FIG. 5 illustrates the repair strap of FIG. 1 used in a repair of a soft tissue and bone interface, according to embodiments of the present invention.

FIG. 6 illustrates a view of the repair of FIG. 6, taken along arrow VI of FIG. 5, according to embodiments of the present invention.

FIG. 7 illustrates a view similar to the view of FIG. 6, illustrating a variation of the installation of the repair strap, according to embodiments of the present invention.

FIG. 8 illustrates an alternative fixation mechanism according to embodiments of the present invention.

FIG. 9 illustrates another alternative fixation mechanism, according to embodiments of the present invention.

FIG. 10 illustrates yet another alternative fixation mechanism, according to embodiments of the present invention.

FIG. 11 illustrates yet another alternative fixation mechanism, according to embodiments of the present invention.

FIG. 12 illustrates an alternative repair strap, according to embodiments of the present invention.

FIG. 13 illustrates the repair strap of FIG. 12 used in a repair, according to embodiments of the present invention.

FIG. 14 illustrates the repair strap of FIG. 12 used in a repair, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

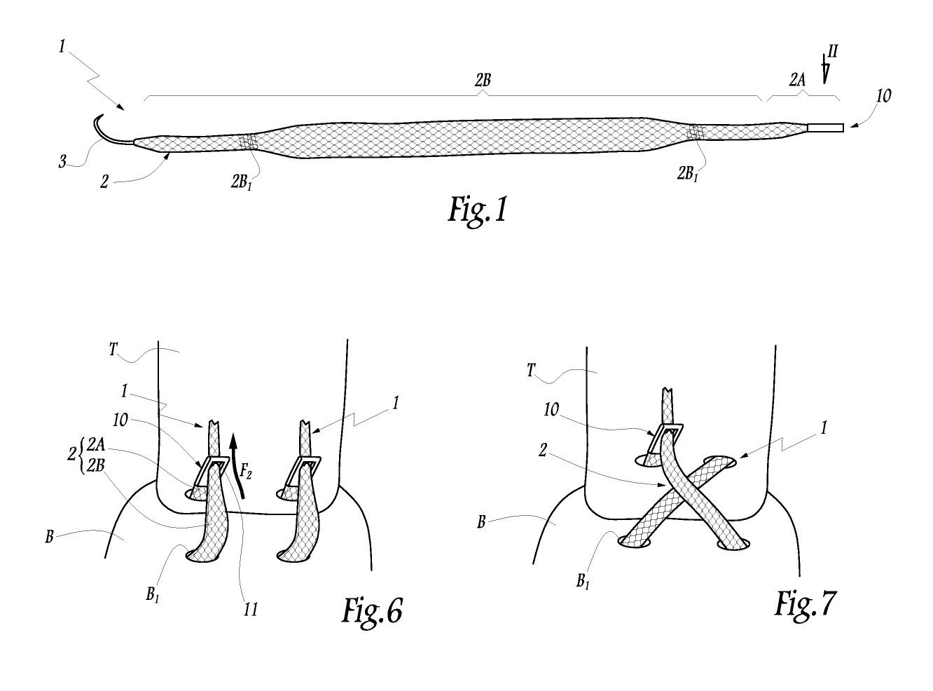

FIG. 1 illustrates a strap 1 adapted to restore the attachment between a soft tissue, such as a tendon or ligament, and a bone, according to embodiments of the present invention. Band 1 includes a flexible elongated textile body 2. According to one embodiment of the present invention, the body 2 is a textile strip, comprising two fibers braided along two different axes. According to an alternative embodiment of the present invention, the fibers of the body 2 define three different axes. Each of the fibers of the strip may be a monofilament or multifilament, rectilinear or twisted in a helical manner around a central axis of a fiber. In some cases, the fibers of the textile strip are made of a resorbable material, for example hydroxybutyrate, Tephaflex®, Artelon®, lactide, and/or glycolide, according to embodiments of the present invention. In other cases, the fibers of the textile strip are made of a non-resorbable material, for example polyester, nylon, polyether ether ketone (PEEK), silk, polyacetal, polyurethane, polyaramide, and the like, according to embodiments of the present invention. According to embodiments of the present invention, the textile body 2 of the strap 1 has a porosity which promotes tissue colonization.

According to one embodiment of the present invention, the textile body 2 has remarkable mechanical characteristics; in other words, the textile body 2 is stronger and stiffer than the soft tissue repaired by the strap 1. In particular, in cases in which the strap 1 is adapted to be used in the human shoulder to repair the attachment of the shoulder bone to the tendons and/or ligaments of the rotator cuff, notably the supraspinatus tendon, the textile body 2 may exhibit a rupture strength greater than 300 Newtons, for example 450 Newtons, and/or a tensile stiffness between fifteen and 300 Newtons per square millimeter, according to embodiments of the present invention. As used herein, the term rupture strength refers to a force required to cause failure of the textile in the pulling-apart or ripping-apart mode.

More generally, the textile body 2 may be adapted to provide a large rupture strength, for greater mechanical resistance and to withstand the forces, as well as to provide a tensile stiffness close to that of the soft tissue to be repaired. The stiffness may be between 0.2 and 2 times that of the soft tissue to be repaired, for example between 0.5 and 1 times that of the soft tissue to be repaired; in this way, the tissue colonization of the textile body 2 is promoted, according to embodiments of the present invention.

As illustrated in FIG. 1, the body 2 of the strap 1 includes two longitudinal parts 2A and 2B along the longitudinal direction of the body. In the example shown, the part 2A is at one end of the body 2, and includes at its free end a rigid or semi-rigid attachment mechanism 10. Part 2B includes the rest of the body 2, including the middle portion and the opposite end portion, according to embodiments of the present invention.

As illustrated in FIG. 1, part 2B of the body 2 includes, near each of its longitudinal ends, a reinforced region 2B₁ at which the longitudinal cross-sectional area changes. For example, the material of the body 2 may be folded back onto itself and/or crushed or welded at regions 2B₁, such that a central region of the body 2 has a larger diameter and/or cross-sectional area than the adjacent regions. This arrangement facilitates manipulation of the end zones of the body 2, while permitting the central portion of the body 2 to have a larger cross section for better reinforcement.

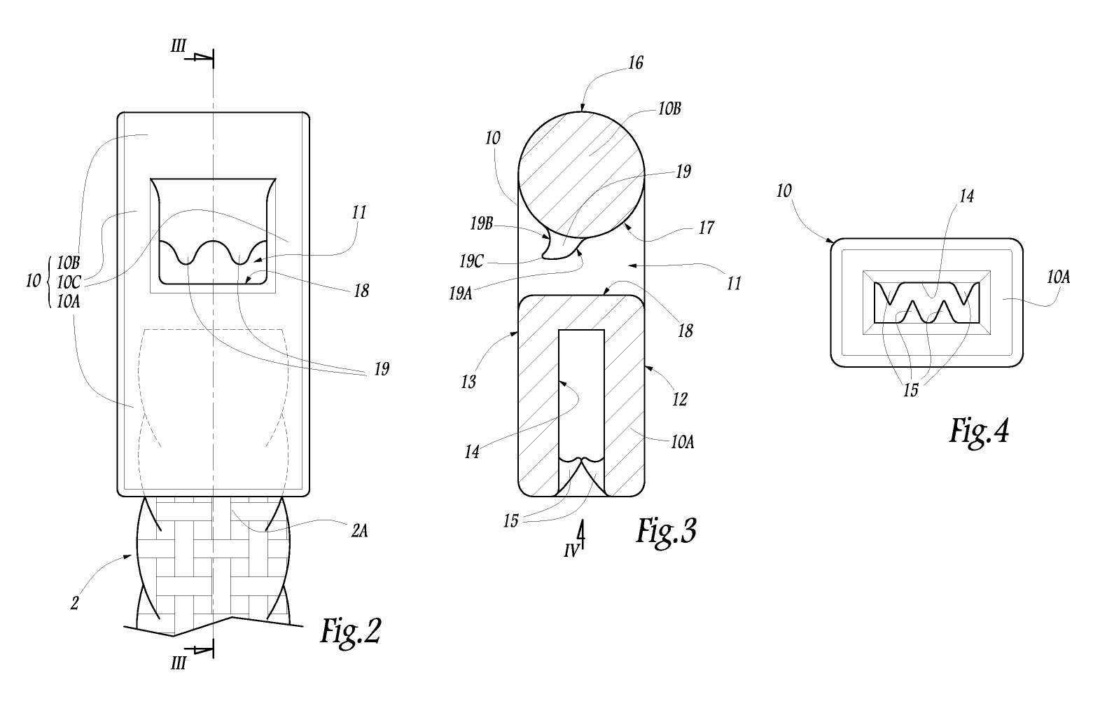

The fixation mechanism 10 at the free end of part 2A of the body 2 is shown in greater detail in FIGS. 2 to 4. Fixation mechanism 10 is a paralleliped whose longer dimension is substantially along the longitudinal direction of body 2, according to embodiments of the present invention. Along the longitudinal direction, fixation mechanism 10 includes two opposing end pieces 10A and 10B, which are connected to each other by two lateral opposed walls 10C, with a hole 11 formed therein, while permitting the central portion of the body 2 to have a larger cross section for better reinforcement.

The fixation mechanism 10 at the free end of part 2A of the body 2 is shown in greater detail in FIGS. 2 to 4. Fixation mechanism 10 is a paralleliped whose longer dimension extends substantially along the longitudinal direction of body 2, according to embodiments of the present invention. Along the longitudinal direction, fixation mechanism 10 includes two opposing end pieces 10A and 10B, which are connected to each other by two lateral opposed walls 10C, with a hole 11 formed therein. The hole 11 extends transversally to the longitudinal direction of the body 2, between the two opposing faces 12, 13 of the fixation mechanism 10.

The base 10A of the fixation mechanism 10, which is oriented toward the body 2, forms an interior cavity 14 which receives and joins to part 2A of the body 2. The cavity 14 is dimensioned in a way that is complementary to the free end of the part 2A, which also permits the end 2A to be introduced in and housed within the cavity 14, according to embodiments of the present invention. The cavity 14 includes one-way grippers or hooks 15, as shown in FIGS. 3 and 4. Each of the points 15 may be formed in the shape of a shark's fin, with the tip of each fin 15 curved inwardly toward the inside of the cavity 14. In the direction facing out from the cavity 14, the fins 15 include a rap inclined toward the interior of the cavity 14, being lightly rounded, with the opposite side of each fin 15 forming a catch which may be lightly hollowed out. In this way, when the part 2A of the body 2 is introduced within the cavity 14, the part 2A slides easily against the ramped surface of the points 15 until it passes over the summits of the points 15, in a direction toward the bottom of the cavity 14. The disengagement of the part 2A from the cavity 14 is mechanically blocked or resisted by the blocking or catching properties of the points 15, which snag or otherwise hinder the reverse movement or pulling out of the part 2A from the cavity 14, according to embodiments of the present invention.

As illustrated in FIG. 4, the one-way hooks 15 may be spaced regularly along the larger dimension of the transverse section of the cavity 14, and may be arranged in staggered or alternative configuration on each opposing face of the cavity 14.

The end piece 10B of the fixation mechanism 10 includes a curved, and/or convex, exterior surface, which helps to minimize potential trauma caused by the locking mechanism 10 after implantation of the strap 1.

As shown in FIGS. 2-4, the end piece 10B of the locking mechanism 10, is shaped like a cylindrical bar with a circular cross sectional shape, centered on an axis perpendicular to the longitudinal direction of the locking mechanism 10, with the aforementioned curved surface 16 corresponding to a portion of the surface of such a cylindrical bar. The portion 17 of the exterior surface of such a cylindrical bar, diametrically opposed to the curved surface 16, at least partially forms a traversing hole 11, as is seen in FIG. 3. With respect to such cylindrical bar, the hole 11 is bounded by a flat surface 18 formed by the end part 10A.

The hole 11 is provided on its interior with one-way hooks or grippers 19, as shown in FIGS. 2 and 3. According to the example shown in the figures, these one-way hooks 19 extend as a projection from the surface 17 of the end part 10B, and may be formed in the shape of a shark fin, according to embodiments of the present invention. The side of the points 19 directed toward primary face 12 of the fixation mechanism 10 forms a ramp which may be slightly curved, while the side of the points 19 directed toward the opposite face 13 forms a hook shape, which may be slightly hollowed out, according to embodiments of the present invention. In this way, when a flexible element is inserted into the hole 11, from the face 12 of the fixation mechanism 10 in a direction toward face 13, the flexible element slides against the surface of the ramp 19A of the points 19 until clearing the summit 19C of the points, after which, in a direction opposite to the insertion, the flexible element is mechanically blocked by the hook or barb shape formed by the back surface 19B and the point 19C. These anti-pullout features 19 retain the flexible element and substantially prevent the withdrawal of the flexible element in a direction opposite to its insertion direction through the hole 11, according to embodiments of the present invention.

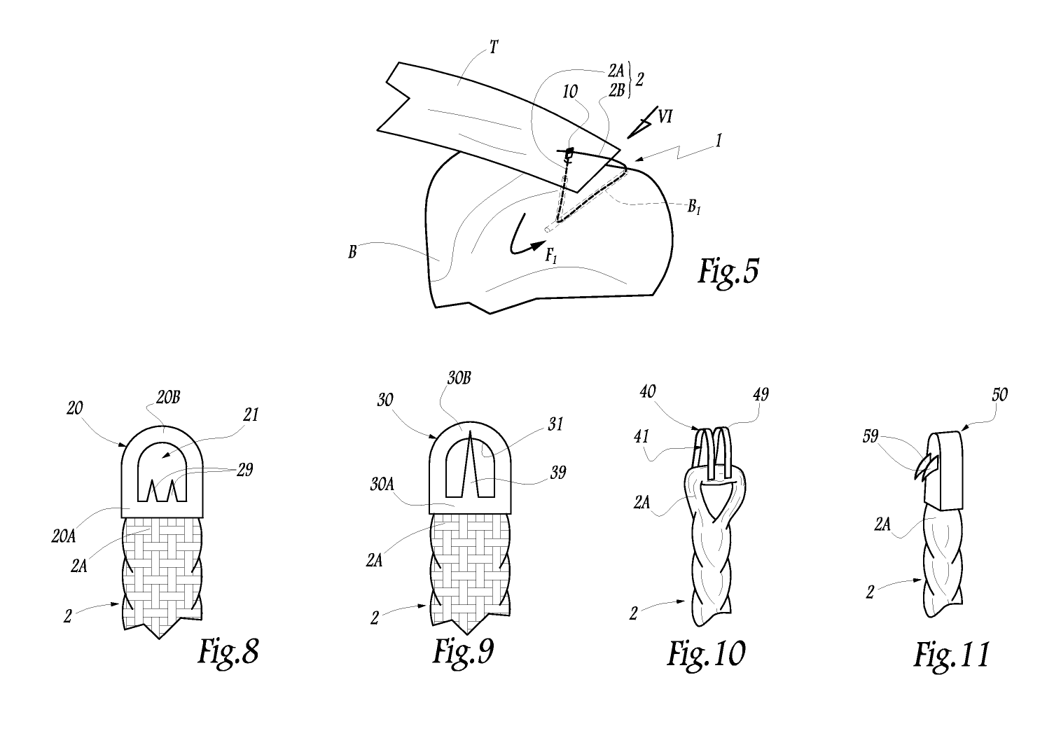

Use of the strap 1 will now be described, in particular with respect to FIGS. 5 and 6, as used to repair the attachment of a soft tissue T to a bone B. As one example, the bone B may be a human humerus while the soft tissue T is one of the tendons or ligaments belonging to the rotator cuff with which the humerus is associated.

Prior to placement of the strap 1, a tunnel B₁ may be formed through the bone B, in the area of its end where attachment of the soft tissue T is desired. As indicated in FIG. 5, the bone tunnel B1 may be drilled or bored at each of its ends into substantially the same face of the bone B, using a drill or boring tool or its surgical equivalent. For example, two different tunnels may be bored into the bone B in different directions in a manner such that the two different tunnels meet each other to form a single tunnel. Alternatively, a tunnel may be drilled straight through the bone B from one surface to another, according to embodiments of the present invention. According to yet other alternative embodiments of the present invention, more complex bone tunnels may be created. The bone tunnel B₁ is created in an area of the bone B which is strong and stable, in order to better support attachment to and force transmission from the soft tissue T, according to embodiments of the present invention.

After formation of the bone tunnel B₁, the free end of the part 2B of the body 2 of the strap 1 may be inserted into the inside of the bone tunnel B₁, and also passed through the soft tissue T. The part 2B may be passed through the tissue T before and/or after its passage through the bone tunnel B₁. To facilitate the insertion of the part 2B through the soft tissue T, as well as to guide part 2B into and through the interior of the bone tunnel B1, the free end of the part 2B may be supplied with an attached needle 3, as illustrated in FIG. 1, or a similar element. In any case, the weaker or smaller cross-sectional area of the end of the body 2B facilitates the threading of that part through the soft tissue T and the bone tunnel B₁, according to embodiments of the present invention.

The free end of part 2B may be threaded through the bone tunnel B1 until it exits the bone tunnel B₁ on the other end, as indicated by arrow F₁ of FIG. 5. As illustrated in FIG. 6, the free end of part 2B emerges as well from the bone B. Free end of part 2B is then threaded through the hole 11 in the fixation mechanism 10, from face 12 to face 13, as indicated by arrow F₂ of FIG. 6, according to embodiments of the present invention.

The needle 3 may facilitate threading of the strap 1 through the hole 11, according to embodiments of the present invention. The body 2 of the strap 1 may also form a loop which is closed onto itself at fixation mechanism 10, encircling or surrounding a part of the soft tissue T and a part of the bone B, as illustrated in FIG. 5. The loop formed by part 2B passing through the hole 11 may be progressively tightened around the tissue T and the bone B; the body 2 is then progressively tensioned along its longitudinal direction, tightening the soft tissue T and the bone B. Because of the one-way fins 19, the loosening of the loop and/or the loss of tension around the soft tissue T and bone B is minimized, according to embodiments of the present invention. As such, the loop formed by the strap 1 may be tightened little by little until the tension is considered sufficient by the surgeon. The extra length of the strap 1 protruding beyond the fixation mechanism 10 may be cut and discarded, according to embodiments of the present invention.

The strap 1 retains the soft tissue T under tension with respect to the bone B, in a manner that is firm and stable. Because the strap 1 is passed through the soft tissue T, then formed into a loop tightened around a part of the soft tissue T, the interaction between the strap 1 and the soft tissue T is solid, and supports elevated tension forces, without the need for addition of any other fixation mechanism between the body 2 and the soft tissue T, the forces being effectively transmitted from the textile body 2 to the soft tissue T, according to embodiments of the present invention. In the same manner, because the strap 1 is passed through a part of the bone B, in forming a loop tightened around that part of bone, the strap 1 is firmly connected to the bone B, without the need for the addition of any other fixation mechanism between the body 2 and the bone B, according to embodiments of the present invention.

Tensioning the soft tissue T with the textile body 2 promotes the rapid and complete tissue in-growth into the textile body 2, according to embodiments of the present invention. Also, the textile body 2 may be configured to maintain this porosity even when formed into a loop and tensioned, according to embodiments of the present invention. Via such tissue in-growth and colonization, the transmission of forces between the body 2 and the soft tissue T is improved, with a distribution that evolves over time, as the healing between the bone B and tissue T interface progresses.

As illustrated in FIG. 6, several straps 1 may be used, in a juxtaposed manner, to reinforce the repair of the attachment between the soft tissue T and bone B. As illustrated in a variation in FIG. 7, the same strap 1 may be used to form two crossed loops, or a “FIG. 8” type loop, which passes through the soft tissue T twice and through the bone tunnel B₁ once, according to embodiments of the present invention. Such straps 1 may be longer than straps which are used to pass through the tissue T only once, according to embodiments of the present invention.

FIGS. 8 through 11 illustrate various embodiments of fixation mechanisms, according to embodiments of the present invention. FIG. 8 illustrates a fixation mechanism 20 which functions similarly to fixation mechanism 10 to repair the attachment of a soft tissue to a bone. Compared with fixation mechanism 10, the fixation mechanism 20 defines a through-hole 21, which functions similarly to hole 11, and is positioned between two ends 20A, 20B of mechanism 20. The part 20A of mechanism 20 is smaller in longitudinal dimension than part 10A, due to the different attachment of the mechanism 20 to the body 2 of the strap 1, according to embodiments of the present invention. For example, the part 2A of body 2 may be glued, sewn, welded, interwoven, or more generally solidly connected in other ways with the end piece 20A of the mechanism 20.

The end 20B of mechanism 20 may be formed as an arc, in a geometrical form different from that of the cylindrical bar of end 10B of the mechanism 10. Numerous other configurations for the end of the fixation mechanism are possible, according to embodiments of the present invention.

The hole 21 may also be provided with points 29 which function similarly to points 19 of mechanism 10. While points 29 extend from the end 20A of the mechanism, points 19 extend from the end 10B of the mechanism 10. This illustrates the numerous possible positions and arrangements for the points, fins, or barbs which prevent loosening of the resulting loop, according to embodiments of the present invention. According to another embodiment of the present invention, the hole 21 may also include points 19 extending into the hole 21 from the top end 20B in addition to or instead of the points 29.

As illustrated in FIG. 9, the body 2 of the strap 1 is integrally provided with a different fixation mechanism 30 which defines a hole 31 surrounded by ends 30A and 30B, which is functionally similar to the hole 21 and to parts 20A and 20B. Instead of points 29, the mechanism 30 includes a tongue 39 extending from end 30B. A zone of flexible deformation where the tongue 39 meets the end 30A permits the tongue 39 to act as a hinge such that, once the loop formed by the body 2 is tightened with the desired intensity, the free end of the tongue 39 blocks the part of the body 2B against the end 30B via pinching. According to another embodiment of the present invention, the body 2 after forming a loop may be tightened with locking mechanism 30 similarly to the tightening of a belt buckle, in which the tongue 39 pierces the body 2 when the loop is tightened to the desired degree.

FIG. 10 illustrates a fixation mechanism 40 including a two rings, or a hole 41 and a ring 49, which may be attached to end 2A, according to embodiments of the present invention. The ring of mechanism 40 is associated with the second ring 49 which may be structurally similar to the ring of mechanism 40. In order to fasten the body 2 back onto itself, the end 2B may be inserted through both rings and then around the second ring and back through the first ring, in order to form a cinch-type mechanism. The end 2B may be coupled with the ring mechanism 40 in various other ways to form a loop which is tightenable but which resists loosening, according to embodiments of the present invention.

FIG. 11 represents another variation of a fixation mechanism 50, which includes barbs or hooks or tines or claws 59 which attach the body 2 of the strap back onto itself to form a loop, according to embodiments of the present invention. The fixation mechanism 50 may be attached to the end 2A of body 2 in various ways, similar to those described above, including but not limited to gluing, crimping, welding, pinning, and the like. The barbs or hooks 59 anchor themselves with respect to the body 2 by penetrating (for example, by “snagging”) the material forming the part 2B of body 2 once the loop formed by the body 2 has been tightened to the desired tension and/or diameter. The hooks 59 firmly maintain mechanical engagement with the part 2B of the body 2 when the body 2 is placed in tension, according to embodiments of the present invention.

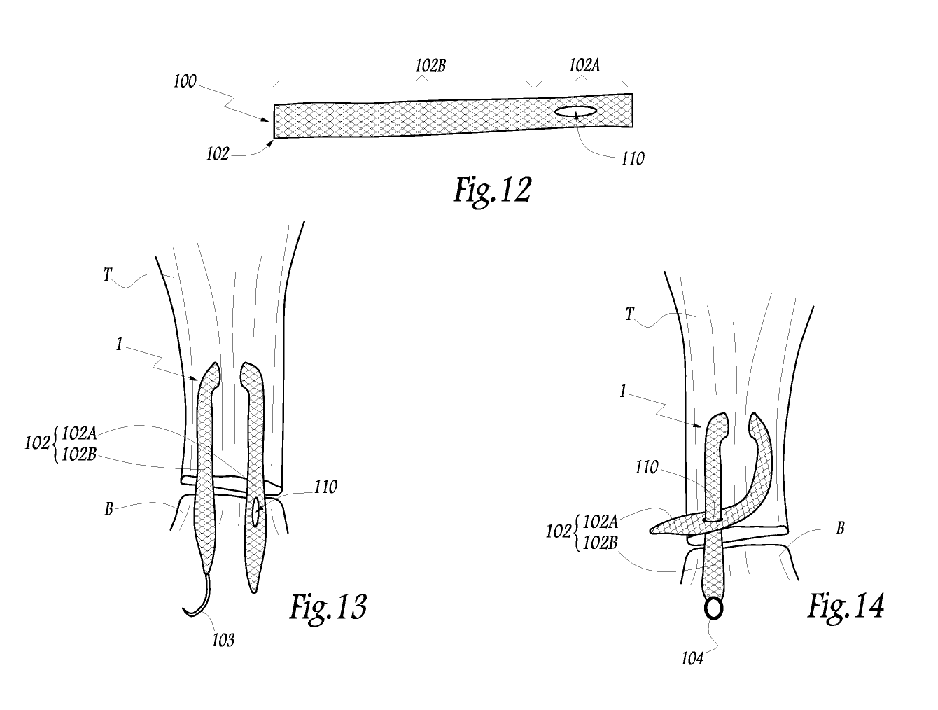

FIGS. 12 to 14 illustrate an alternative embodiment of a strap 100 adapted to repair the attachment of a soft tissue T, such as a tendon or a ligament, to a bone B. The strap 100 includes a flexible elongated body 102 which is structurally and functionally similar to body 2 of strap 1, according to embodiments of the present invention. Strap 100 permits the attachment of its two different longitudinal components 102A and 102B to each other to form a loop closed on itself. The part 102A of the body 102 includes a slit 110 dimensioned to permit the end 102B of body 102 to be introduced therethrough, after end 102B has been passed through tissue T and/or a bone tunnel B and a loop formed, the loop passing through and/or encircling a portion of the tissue T, according to embodiments of the present invention.

The slit 110 may include a simple form, for example a longitudinal cut in the material of which the part 102A is formed. In one alternative example (not shown), the border of the slit 110 may be reinforced with one or more pieces of rigid or semi-rigid material to form an eyelet. Such an eyelet may be provided with one or more mechanical fixation mechanisms functionally and/or structurally similar to the anti-loosening fins, barbs, or hooks, 19, 29 or the tongue 39 described above, which may also be referred to as locking elements or mechanical locking elements, according to embodiments of the present invention. The slit 110 permits the fixation of part 2B of the body 2 to part 2A of the body 2 to be tensioned to form a loop. Strap 100 may be used in a fashion similar to that of strap 1, as described above.

FIGS. 13 and 14 illustrate an alternative embodiment of the utilization of the strap 100, which may also be employed with strap 1. Instead of passing the part 102B through both the tissue T and a bone tunnel B₁, part 102B is passed only through the tissue T, for example with the help of a needle 103 similar to needle 3, after which the part 102B is threaded through the slit 110 of part 102A. Next, the loop formed by the body 102 is tightened with the desired intensity, and the part 102B of the body 102 is attached to the bone B by a fixation element 104, such as a screw, or a bone anchor, as illustrated in FIG. 14, according to embodiments of the present invention. As an alternative (not shown) to the configuration of FIG. 14, the end 102A may be anchored to the bone B and the end 102B may be left free, such that the loop formed by the body 102 may be tightened by the surgeon even after end 102A has been anchored to the bone, by pulling on the free end 102B to tighten the loop. According to such embodiments, the free end 102B may then be tied in a knot or otherwise secured with respect back to the body 102, or to the bone B.

Various arrangements and variations of the straps 1 and 100 and the related methods of use or implantation may be employed. For example:
The directional arrangement between the ends 2A or 102A and the holes 11, 21, 31, 41, or the slit 110 are not limited to that which is shown;
After situating the strap 1 or 100, the strap can be sutured to the soft tissue T, in particular to avoid the small movements or vibrations or abrasions therebetween which, over time, can cut the body 2 or 102;
The textile body 2 of the strap may, in addition to the mechanical properties of rupture strength and tensile stiffness described above, permit a suture pull-out resistance greater than 150 Newtons;
The straps 1 or 100 may be used in either open surgery and/or endoscopic procedures;
The parts 2B or 102B may, at least locally, be coated with a lubricant, sheathed, or surface treated in order to facilitate their sliding across the holes 11, 21, 31 and 41 or the slit 110, as well as across the soft tissue T and, where necessary, the bone tunnel B₁ formed across the bone B;
The material of which the body 2 is formed may be adapted, in particular after having passed the body 2 through the soft tissue T and, when necessary, the bone B, to include protruding transversal elements which cooperate mechanically with the locking between the part 2B or 102B and the holes 11, 21, 31, 41 or the slit 110; and/or
As illustrated in FIGS. 5, 6, 13, and 14, the strap 1 or 100 may be used solely for attaching the soft tissue T to the bone B, or, as a variation, the strap may be used in conjunction with a patch which the strap maintains against the soft tissue T, for example in order to cover an extended area of tissue and/or to further distribute the forces of the strap 1 or 100 over a larger surface area of the tissue. For example, the strap 1 or 100 may be used in conjunction with or as part of a patch similar to those described in U.S. Patent Application Publication Number 2008/0188936, published on Aug. 7, 2008, which is incorporated by reference herein in its entirety for all purposes.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A strap for repairing the attachment between a bone and a soft tissue, comprising:
an elongated flexible textile body comprising, along its longitudinal direction, first and second longitudinal parts, the first longitudinal part comprising a fixation mechanism, the fixation mechanism configured to mechanically cooperate with the second longitudinal part, after the second longitudinal part has been passed through at least the soft tissue, to form a loop around at least a portion of the soft tissue, wherein the fixation mechanism permits tightening of the loop to tension the elongated flexible textile body to hold the soft tissue in place with respect to the bone.

2. The strap of claim 1, wherein the bone is a shoulder bone, and wherein the soft tissue comprises rotator cuff tendons and ligaments.

3. The strap of claim 1, wherein the elongated flexible textile body comprises a porosity that permits tissue in-growth even when the elongated flexible textile body is under tension.

4. The strap of claim 1, wherein the elongated flexible textile body has a maximum rupture strength greater than 300 Newtons.

5. The strap of claim 4, wherein the elongated flexible textile body has a maximum rupture strength greater than 450 Newtons.

6. The strap of claim 1, wherein the elongated flexible textile body has a stiffness between fifteen Newtons per millimeter and 300 Newtons per millimeter.

7. The strap of claim 1, wherein the elongated flexible textile body exhibits a suture pull-out strength greater than 150 Newtons.

8. The strap of claim 1, wherein the elongated flexible textile body comprises braided fibers.

9. The strap of claim 1, wherein the fixation mechanism comprises a hole configured to receive the second longitudinal part.

10. The strap of claim 1, wherein the fixation mechanism comprises a rigid or semi-rigid element which at least partially defines a hole, the rigid or semi-rigid element being fixedly attached to a free end of the elongated flexible textile body.

11. The strap of claim 9, wherein the hole is a slit arranged across material forming the first longitudinal part.

12. The strap of claim 9, wherein the fixation mechanism comprises a mechanical locking element adapted to retain the second longitudinal part within the hole and to resist loosening of the loop.

13. The strap of claim 12, wherein the locking element is positioned within the hole and adapted to engage the second longitudinal part.

14. The strap of claim 12, wherein the locking element is adapted to pinch the second longitudinal part against the fixation mechanism.

15. The strap of claim 1, wherein the fixation mechanism comprises one or more claws configured to penetrate a portion of material out of which the second longitudinal part is formed.

16. The strap of claim 1, wherein the fixation mechanism comprises a cavity configured to receive a free end of the first longitudinal portion, wherein the cavity comprises one or more locking elements configured to prevent disengagement of the free end from the cavity.

17. A method for attaching a tissue to bone, comprising:
passing a flexible elongated textile body through the tissue, the flexible elongated textile body comprising a fixation mechanism;
connecting the flexible elongated textile body onto itself with the fixation mechanism to form a loop;
engaging the flexible elongated textile body with the bone; and
narrowing the loop to tension the flexible elongated textile body to maintain the portion of the tissue in place with respect to the bone.

18. The method according to claim 17, wherein the flexible elongated textile body has an ultimate stress property higher than the tissue.

19. The method according to claim 17, wherein engaging the flexible elongated textile body with the bone comprises passing the flexible elongated textile body through a bone tunnel in the bone.

20. The method of claim 17, wherein passing the flexible elongated textile body through the bone tunnel comprises passing the flexible elongated textile body through the bone tunnel before the loop is formed.

21. The method of any of claims 17, wherein passing the flexible elongated textile body through the tissue comprises passing the flexible elongated textile body through the tissue a first time, the method further comprising passing the flexible elongated textile body through the tissue a second time.

22. The method according to claim 17, wherein passing the flexible elongated textile body through the tissue comprises passing the flexible elongated textile body through the tissue at a first location, wherein the bone tunnel is a first bone tunnel, the method further comprising passing the flexible elongated textile body through the tissue at a second location and passing the flexible elongated textile body through a second bone tunnel in the bone.

23. The method of claim 22, wherein passing the flexible elongated textile body through the tissue at the first and second locations and through the first and second bone tunnels occurs before the flexible elongated textile body is connected onto itself with the fixation mechanism.

24. The method of claim 17, wherein the flexible elongated textile body is a first flexible elongated textile body and wherein the bone tunnel is a first bone tunnel, the method further comprising passing a second flexible elongated textile body through the tissue and through a second bone tunnel in the bone.
